(19) United States
(12) Patent Application Publication
(10) Pub. No.: US 2025/0281203 A1
(43) Pub. Date: Sep. 11, 2025

(54) INSEMINATION DEVICE FOR MEDICALLY ASSISTED PROCREATION

(71) Applicant: VESLA S.R.L., ROMA (IT)

(72) Inventor: Sara Fusco, Roma (IT)

(73) Assignee: VESLA S.R.L., ROMA (IT)

(21) Appl. No.: 18/863,827

(22) PCT Filed: May 10, 2023
(86) PCT No.: PCT/IB2023/054836

§ 371 Date: Nov. 7, 2024

(30) Foreign Application Priority Data
May 11, 2022 (IT) 102022000009731

(51) Int. Cl. A61B 17/43; A61B 17/00

(52) U.S. Cl. CPC A61B 17/43; A61B 2017/00115; A61B 2017/00367; A61B 2017/00477; A61B 2017/00544

(57) ABSTRACT

An insemination device for an in vitro fertilisation procedure; said insemination device comprising a pneumatic actuator and a needle-holder, which is connected to said pneumatic actuator by means of a pneumatic transmission tube; said insemination device being characterized in that it comprises a thermostated magazine arranged between said needle-holder and said pneumatic transmission tube, and wherein a plurality of tanks are obtained, each designed to accommodate at least one spermatozoon; said magazine being designed to be moved so as to selectively cause each one of said tanks to simultaneously establish a fluid-dynamic connection both to said needle-holder and to said pneumatic transmission tube.

### CROSS-REFERENCE TO RELATED APPLICATIONS

This patent application claims priority from Italian patent application no. 102022000009731 filed on May 11, 2022, the entire disclosure of which is incorporated herein by reference.

### TECHNICAL FIELD

The present invention relates to an insemination device for carrying out the procedure of Medically Assisted Procreation (MAP).

### BACKGROUND

In MAP, the insemination procedure envisages inserting the spermatozoon into the egg cell using the Intracytoplasmic sperm injection (ICSI) technique.

ICSI is a technique born about 30 years ago and envisages that fertilisation takes place with the mechanical introduction of the spermatozoon into the decumulated oocyte, that is, deprived of the cells of the cumulus and the radiant crown.

The ICSI technique, by providing that the spermatozoon is inserted into the cytoplasm, has the advantage of also being able to treat oligoasthenoteratozoospermia cases, i.e. seminal fluids with reduced concentration, motility and morphology.

In addition, this technique guarantees the activation of the oocyte, as the ovarian cytoplasm is aspirated into the glass pipette that carries the spermatozoon. In this way, the contact between the ovarian cytoplasm and the spermatozoon and, therefore, the activation of the oocyte, are promoted.

Despite the above advantages, the ICSI technique suffers from the disadvantage of still being extremely dependent on the capabilities of the operator with regard to the oocyte insemination times.

During the ICSI procedure the oocytes are placed in an injection dish, inside which microdrops of buffered culture medium and covered with liquid paraffin oil preheated to a temperature of 37° have been prepared. Under these conditions, the operator is expected to carry out the insemination in a time ranging from one and a half to two minutes per oocyte. Since the manual skills of the operators are very varied, it can happen that an oocyte gets inseminated in longer times thereby entailing the risk of thermal and oxidative stress that might have an impact on the development of the embryos. In addition, taking into account that normally six oocytes are inseminated at a time (arranged two by two in three microdrops), the time between the first and the last inseminated oocyte is between ten and twelve minutes. This is a risky time frame that can have negative effects on fertilisation, cleavage (cell division), and blastulation.

For thirty years now, the device used to collect one spermatozoon at a time and inseminate the oocyte has not undergone any significant changes. In fact, despite the technological improvement, the lack of standardization of the results in terms of fertilisation still remains. The two essential variables that influence the outcomes of the in vitro fertilization are the skill of the operator and the quality of the oocytes to be inseminated. While there are major obstacles to the quality of the oocytes in medicine, since no action can be taken on the limits related to genetics or to the biological age, it is possible to intervene on the quality of the procedure in terms of technological progress.

According to the technique used to date, the operator must collect a spermatozoon from the polyvinylpyrrolidone drop with the micropipette and then move in the drop with the oocytes and inseminate the first oocyte. The same step must be repeated for as how many oocytes there are to inseminate.

The thermal stress of the cells and the formation of free radicals is directly proportional to the exposure time outside the incubator. In fact, the main cause of oxidative stress is oxygen and therefore the exposure of the oocytes to atmospheric oxygen induces a reduction in the development at the stage of morula and blastocysts. Various studies have demonstrated that these effects are irreversible and remain even if, subsequently, the oocytes are placed in the incubators at optimal oxygen concentrations and temperature levels.

Gametes and embryos are natural sources of free radicals, but during manipulation there is an increased risk of generating super-physiological levels by inducing a state of oxidative stress that can have a significant impact on the in vitro fertilisation.

In conclusion, it is clear from the above that a prolonged exposure to the atmospheric oxygen can induce an increase in oxidative stress and, consequently, a reduction in the fertilisation rate, thus compromising the pregnancy outcome.

The need was therefore felt for an instrument that reduced the time during which the oocytes are exposed to the atmospheric oxygen and, at the same time, reduce the dependence of the success of the in vitro fertilisation technique on the capabilities of the operator.

The inventor of the present invention has realized a device, the technical characteristics of which are such as to satisfy the above needs.

### SUMMARY

The subject-matter of the present inventions is an insemination device for an in vitro fertilisation procedure; said insemination device comprising a pneumatic actuator and a needle-holder, which is connected to said pneumatic actuator by means of a pneumatic transmission tube; said insemination device being characterized in that it comprises a thermostated magazine arranged between said needle-holder and said pneumatic transmission tube, and in which a plurality of tanks are obtained, each designed to accommodate at least one spermatozoon; said magazine being designed to be moved so as to selectively cause each one of said tanks to simultaneously establish a fluid-dynamic connection both to said needle-holder and to said pneumatic transmission tube.

Here and hereinafter “fluid-dynamic connection” means that a fluid flowing channel is realized between the needle, one of the tanks and the pneumatic transmission tube.

Preferably, each of said tanks is a tube with a capillary diameter ranging from 400 to 800 μm.

Preferably, said device comprises a support structure, which houses said magazine and comprises said needle-holder as well as a coupling duct designed to be connected to said pneumatic transmission tube; said needle-holder and said coupling duct being aligned with one another.

Preferably, said magazine is a drum, which is suited to rotate around a longitudinal symmetry axis of its and wherein a plurality of cylindrical holes are obtained, which are longitudinally arranged around said longitudinal symmetry axis and are each designed to define a respective tank; each one of said cylindrical holes being designed to be selectively aligned with and establish a fluid-dynamic connection to said needle-holder and said coupling duct.

Preferably, said support structure comprises two parts hinged to one another so as to allow access to said magazine from the outside.

Preferably, said support structure comprises electric moving means designed to move said magazine.

Preferably, said device comprises a control system, which is designed to detect a previously set condition of the pneumatic actuator and is connected to at least one visual and/or acoustic signaller.

Preferably, said device comprises an indicator which informs on the tank involved in the in vitro fertilisation operations.

### BRIEF DESCRIPTION OF THE DRAWINGS

Hereinafter an embodiment is reported for illustrative and non-limiting purposes with the aid of the accompanying Figures, wherein:

FIG. 1 shows a device according to the present invention with in section and with parts in transparency for a better understanding; and

FIG. 2 shows part of the device of FIG. 1 in a different operating conformation.

### DESCRIPTION OF EMBODIMENTS

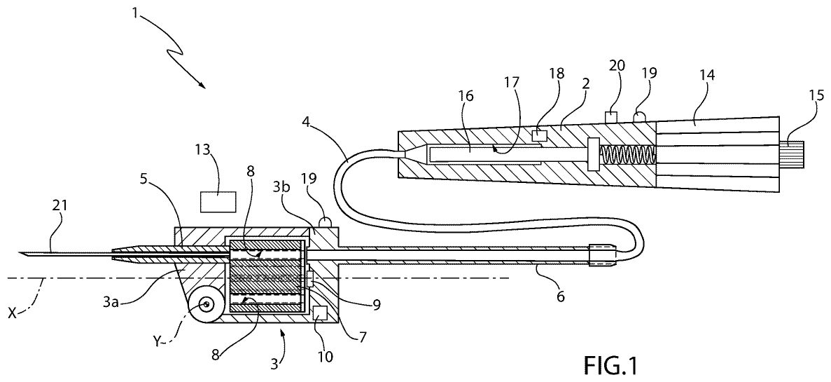

In FIG. 1, 1 denotes as a whole the device subject-matter of the present invention, according to a preferred embodiment.

The device 1 comprises a pneumatic actuator 2 and a support structure 3 connected to the pneumatic actuator 2 by means of a pneumatic transmission tube 4. The support structure 3 comprises a needle-holder 5 and a coupling duct 6 extending on the opposite side with respect to the needle-holder 5 and which is designed to be connected to one end of the pneumatic transmission tube 4. In particular, the needle-holder 5 and the coupling duct 6 define respective channels aligned with one another.

The device 1 comprises a drum-shaped magazine 7 housed in the support structure 3 where it is free to move around its own longitudinal symmetry axis X.

A plurality of through holes 8 are obtained in the magazine 3 which are arranged with an axis parallel to the axis X and symmetrically around the axis X. Each of the through holes 8 defines a tank for housing at least one spermatozoon to be inserted inside an oocyte in an in vitro fertilisation procedure. By means of the rotation of the magazine 7 about the axis X, each of the through holes 8 will be selectively in an operating position and aligned both with the needle-holder 5 and with the coupling duct 6. The support structure 3 comprises an encoder schematically shown and denoted with 9, which engages a portion of the magazine 7 to cause its rotation around the axis X and selectively arrange each of the through holes 8 in their operating position. A display 10 connected to the encoder 9 is arranged in the support structure 3. The display 10 informs on which through hole 8 is the one that is in the operating position (aligned with the needle-holder 5 and with the coupling duct 6).

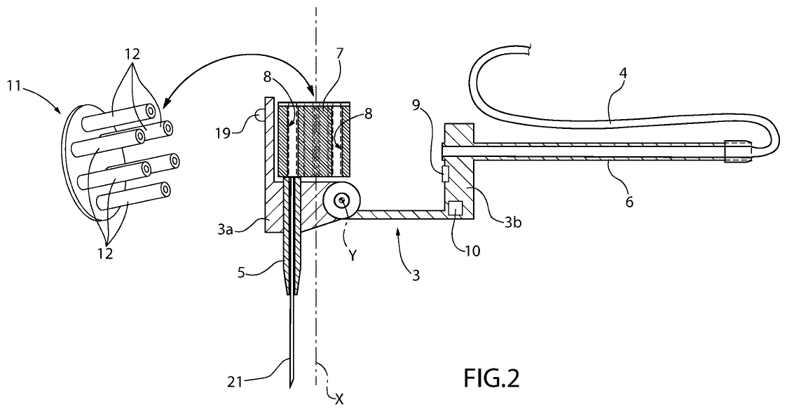

The support structure 3 is composed of a front part 3a and a rear part 3b that are hinged to one another along an axis Y that is arranged orthogonal to the axis X. The rotation along the axis Y implies that the support structure can be arranged both in a closed conformation (FIG. 1), wherein between the front part 3a and the rear part 3b there is defined the housing of the magazine 7, and in an open conformation (FIG. 2), wherein access from the outside of the through holes 8 is allowed. The open conformation allows the insertion of a disposable gasket 11, comprising a plurality of tubes 11 each of which will be housed inside a respective through hole 8, so as to define in the respective through hole 8 the external walls of the single tank. Each of the tubes 12 has a micrometric diameter ranging from 100 to 800 μm.

The micrometric dimensions guarantee the permanence of the aspirated fluid inside the single tube.

From what is described above, the structural and functional similarity between the support structure 3 and a drum gun may be immediate to the reader.

The magazine 7 is connected to thermostatting means schematically shown and denoted with 13, useful for maintaining the magazine as a whole at a temperature of about 37° C.

The pneumatic actuator 2 comprises a macrometric knob 14 and a micrometric knob 15, which control with different sensitivity the movement of a piston 16 that slides inside a cylinder 17 obtained inside the same pneumatic actuator 2. The movement of the piston 16 allows both aspiration and injection of a micro-sample as will be described below.

The pneumatic actuator 2 comprises a system for the position control of the piston 16, in order to inform the operator about the progress of the in vitro fertilisation procedure. In particular, the control system is represented by a transmitter chip schematically shown and denoted with 18. The transmitter chip 18 communicates with one or more visual signallers (lights) 19, which are housed on the support structure 3 and/or on the pneumatic actuator 2. Alternatively, or in combination with the visual signallers, the device may also be provided with acoustic signallers.

The insemination device 1 further comprises a button 20 which is arranged on the body of the pneumatic actuator and is connected with the encoder 9 to control the activation thereof and, consequently, the rotation of the magazine 7.

The in vitro fertilisation procedure using the insemination device 1 according to the invention will be described below. The in vitro fertilization procedure consists essentially of two steps: (i) sequential collection of the spermatozoa; (ii) sequential insemination of the oocytes.

The preparation step of the insemination device 1 provides that firstly the thermostatting means 13 are activated to maintain the magazine 7 at a temperature of about 37° C. Subsequently, the support structure 3 is brought into its open configuration so that the disposable gasket 11 can be loaded into the magazine 7. At this point a needle 21 is inserted into the needle-holder 5. The needle 21 will be inserted until it reaches the respective tube 11 with which it is aligned. The needle 21 and the disposable gasket 11 are the only parts that come into contact with human material and that will therefore be replaced at each new in vitro fertilisation procedure.

Once the insemination device 1 has been prepared as reported above, the step of sequential collection of the spermatozoa can be carried out. This step provides for the insemination device to collect spermatozoa which have meanwhile been treated according to the traditional technique, for example with hyaluronic acid or polyvinylpyrrolidone. The aspiration by the insemination device is carried out by means of an aspiration controlled by a suitable rotation of the knobs 14 and 15. During aspiration, the transmitter chip 18 detects when the piston 16 has reached a previously set position such as to guarantee that the single spermatozoon has been arranged by aspiration within a respective tube 12. The transmitter chip 18 activates the visual signallers 19 to indicate that the piston has reached the previously identified position and that, therefore, the spermatozoon has been housed in the tank aligned with the needle 21. At this point, the operator by means of the button 20 commands the encoder 9 to carry out a rotation of the magazine 7 around the axis X, bringing a new through hole 8 and, therefore, a new tube 12, aligned with the needle 21 and with the coupling duct 6. The rotation of the magazine 7 is displayed on the display 11, where the number corresponding to the through hole 8 that is in the operating position is indicated. Once this condition is reached, the above reported operations are repeated, thus leading to a second spermatozoon being housed in a second tank. In particular, each time the magazine 7 rotates to bring a new tube 12 into the operating position, at least one of the visual signallers 18 informs that the same operating position has been reached and a new tube 12 is ready to receive a respective spermatozoon. At the same time, the display reports the number corresponding to the through hole 8 wherein the tube 12 ready to load a new spermatozoon is arranged.

According to a further embodiment, the transmitter chip 18 is connected directly to the encoder 9, such that the magazine 7 is automatically rotated once the transmitter chip 18 itself detects the previously set position of the piston 16.

The above operations are repeated until all the tubes 12 of the magazine 7 are filled.

Once the step (i) of sequential collection of the spermatozoa has been completed, it is moved to step (ii) of sequential insemination of the oocytes.

The oocytes are collected from the incubator and subsequently placed in the microdrops of an injection dish according to the traditional technique.

At this point the insemination device 1 starts the insemination of a first oocyte using the thrust caused by the pneumatic actuator 2 by means of the rotation of the knobs 14 and 15 by the operator. Once the insemination of the first oocyte is ended, the operator, by means of the button 20, commands a rotation of the magazine 7 around the axis X bringing a new through hole 8 and, therefore, a new tube 12, aligned with the needle 21 and with the coupling duct 6. In this new operating position of the insemination device 1, a second oocyte is inseminated. Similarly, to what is reported above for the collection step (i), each time the magazine 7 rotates to bring a new tube 11 into the operating position, at least one of the visual signallers 18 informs that the same operating position has been reached and that, therefore, a new tube 12 “loaded” with spermatozoon is placed to proceed with the insemination of a new oocyte. At the same time, the number assigned to the through hole 9 that is in the operating position is indicated on the display 11.

These operations are repeated until all spermatozoa present in the magazine 7 have been injected into respective oocytes.

As may seem immediate from the above description, the insemination device according to the present invention allows both to drastically reduce the execution time and to automate the operations so as to decrease the influence of the capabilities of the individual operator on the success of the Medically Assisted Procreation procedure as a whole.

The use of the device according to the present invention allows the oocytes to be collected only for the time necessary for their insemination. This allows to significantly reduce the risks of oxidative stress. For example, it has been calculated that with the device subject-matter of the present invention it is possible to carry out the insemination of the single oocyte in a time not exceeding one minute thus allowing to inseminate a maximum of six oocytes in less than five minutes, instead of the ten/twelve minutes of the prior art.

1. An insemination device (1) for an in vitro fertilisation procedure; said insemination device comprising a pneumatic actuator (2) and a needle-holder (5), which is connected to said pneumatic actuator (2) by means of a pneumatic transmission tube (4); said insemination device being characterized in that it comprises a thermostated magazine (7) arranged between said needle-holder (5) and said pneumatic transmission tube (4), and wherein a plurality of tanks (8, 12) are obtained, each designed to accommodate at least one spermatozoon; said magazine (7) being designed to be moved so as to selectively cause each one of said tanks (8, 12) to simultaneously establish a fluid-dynamic connection both to said needle-holder (5) and to said pneumatic transmission tube (4).

2. The insemination device according to claim 1, characterized in that each one of said tanks is a tube (8, 12) with a capillary diameter ranging from 100 to 800 μm.

3. The insemination device according to claim 1, characterized in that it comprises a support structure (3), which houses said magazine (7) and comprises said needle-holder (5) as well as a coupling duct (6) designed to be connected to said pneumatic transmission tube (4); said needle-holder (5) and said coupling duct (6) being aligned with one another.

4. The insemination device according to claim 3, characterized in that said magazine (7) is a drum, which is suited to rotate around a longitudinal symmetry axis (X) of its and in which a plurality of cylindrical holes (8) are obtained, which are longitudinally arranged around said symmetry longitudinal symmetry axis and are each designed to define a respective tank; each one of said cylindrical holes (8) being designed to be selectively aligned with and establish a fluid-dynamic connection to said needle-holder (5) and said coupling duct (6).

5. The insemination device according to claim 3, characterized in that said support structure (3) comprises two parts (3a and 3b) hinged to one another so as to allow access to said magazine (7) from the outside.

6. The insemination device according to claim 1, characterized in that said support structure (3) comprises electric moving means (9) designed to move said magazine (7).

7. The insemination device according to claim 1, characterized in that it comprises a control system (18), which is designed to detected a previously set condition of the pneumatic actuator (2) and is connected to at least one visual and/or acoustic signaller (19).

8. The insemination device according to claim 1, characterized in that it comprises an indicator (10), which informs on the tank involved in the in vitro fertilisation operations.
